(19) United States
(12) Patent Application Publication
(10) Pub. No.: US 2025/0213428 A1
(43) Pub. Date: Jul. 3, 2025

(54) SINGLE-USE FLEXIBLE BAG WITH SUMP

(71) Applicant: AQUASYN, LLC, Carson City, NV (US)

(72) Inventor: Michael C. Gagne, Carson City, NV (US)

(73) Assignee: AQUASYN, LLC, Carson City, NV (US)

(21) Appl. No.: 19/087,378

(22) Filed: Mar. 21, 2025

Related U.S. Application Data
(63) Continuation of application No. PCT/US2023/075117, filed on Sep. 26, 2023.
(60) Provisional application No. 63/377,487, filed on Sep. 28, 2022.

(51) Int. Cl. A61J 1/10; A61J 1/14; A61J 1/20

(52) U.S. Cl. CPC A61J 1/10; A61J 1/1475; A61J 1/2058

(57) ABSTRACT

A flexible bag having one or more side surfaces and a bottom surface that includes a sump region formed by one or more side sump surfaces and a bottom sump surface. A first connecting port is disposed in the bottom sump surface. A second connecting port is disposed in a side sump surface. The first connecting port is connected to a first pump having one or more outlets while the second connecting port is connected to a second pump having one or more outlets. The flexible bag includes a plurality of fluid ports. One or more of the outlets of the first and/or second pump are connected to conduit/tubing that connect to one or more of the fluid ports. Fluid in the flexible bag can then be recirculated back into the flexible bag for mixing and/or agitation in addition to being pumped to downstream processes.

### RELATED APPLICATIONS

This Application is a continuation of International Application No. PCT/US2023/075117 filed on Sep. 26, 2023, which itself claims priority to U.S. Provisional Patent Application No. 63/377,487 filed on Sep. 28, 2022, which is hereby incorporated by reference. Priority is claimed pursuant to 35 U.S.C. §§ 119, 120 and any other applicable statute.

### TECHNICAL FIELD

The technical field generally relates to containers used in connection with biopharmaceutical, pharmaceutical, and biotechnology applications. More specifically, the technical field relates to flexible containers such as bags that hold fluids therein and includes a sump region in a bottom surface that contains port(s) for securing one or more pumps. A plurality of fluid ports are also located in the flexible bag that may be used as fluid return flow paths to recirculate and/or mix fluid.

### BACKGROUND

Biopharmaceutical, pharmaceutical, and biotechnology applications have historically used reusable devices in the research and manufacturing processes associated with these industries. These reusable devices, however, must be cleaned and sterilized between uses. This was typically accomplished using a sterilization agent (e.g., steam) that was introduced into the reusable devices. The sterilization process, however, is time-consuming and introduces considerable cost into the process. Moreover, there is the possibility that sterilization was not effective, leading to contamination issues. More recently, these industries are moving toward using single-use components instead of reusable devices for parts and components that come into contact with reagents and products. An advantage of single-use or disposable products is that it eliminates the need for cleaning and sterilizing devices in between uses.

Containers for holding fluid are regularly used in the biopharmaceutical, pharmaceutical, and biotechnology fields. These containers may be rigid or flexible depending on the particular process or application. Sometimes, fluid within the containers needs to be mixed or agitated. One common way to aid in mixing is to add a mixing unit includes a shaft and impeller that extends downward into the interior of the container and rotates to mix fluid. Flexible fluid containers such as bags are also used in biopharmaceutical, pharmaceutical, and biotechnology applications. Flexible fluid containers may include bags that hold fluids therein. These flexible fluid containers may be single-use products which can be discarded after use. Fluid containers such as bags can be coupled directly or indirectly to pumps to evacuate fluid from the interior of the bag to other processes. There is a need for solutions to incorporate pumping, mixing, and/or agitation into flexible fluid containers such as bags.

### SUMMARY

In one embodiment, a single-use flexible bag is disclosed that includes one or more ports that are used to fill the flexible bag with fluid. One or more of these ports may also act to vent air/gases when fluid is filling the flexible bag. The flexible bag further includes a bottom surface that includes a sump region. The sump region of the flexible bag is located generally at the central region of the bottom surface and further extends in the downward direction (based on the orientation of the a single-use flexible bag when being used). The bottom surface of the flexible bag adjacent to the periphery of the sump region is tapered toward the sump region so that when fluid is evacuated from the main interior region of the flexible bag, fluid will naturally flow (due to gravity) into the sump region. The sump region of the flexible bag includes a bottom sump surface that includes an aperture formed therein. In one embodiment, a port is located in or on the aperture and is used to connect with other components such as, for example, a pump. In one embodiment, the port is a flanged port or a port with a sanitary connector.

In a preferred embodiment, the port in the sump region is coupled to a pump. The pump, in one embodiment, has an inlet (or in some cases multiple inlets) that receives fluid from the flexible bag via the port in the sump region and pumps this fluid out via one or more outlets. An example of the pump that may be coupled to the flexible bag includes the pump or pump/mixer devices disclosed in International Patent Application No. PCT/US2021/015917 published as WO 2021/158448, which is incorporated by reference.

In another embodiment, the sump region contains another port located in one or more sump sidewalls that define the sump region. The one or more sump sidewalls is/are generally perpendicular to the bottom sump surface in one embodiment. Similar to the bottom port of the sump region, this side port is located in or on an aperture in the sump sidewall. In one embodiment, the port is a flanged port or a port with a sanitary connector. In a preferred embodiment, this side port in the sump region is coupled to a separate pump. The separate pump has an inlet (in some embodiments additional inlets may be included in the pump) that receives fluid from the flexible bag via the side port in the sump region and pumps this fluid out via one or more outlets. In one preferred embodiment, one or more of the outlets of this second pump pumps fluid back into the flexible bag as explained herein.

The flexible bag contains a plurality of fluid ports formed therein for receiving fluid from one or more of the sump pumps (or other pumps). This may be the bottom sump pump or the side sump pump. The sump pump may include a multi-outlet pump that includes conduits or tubing that connect at least some of the outlets of the multi-outlet pump to the fluid ports. In some embodiments, the fluid ports may include respective valves incorporated therein to control the flow of fluid into the flexible bag with fluid. The fluid ports, in one embodiment, are located close to the bottom surface of the flexible bag. This includes locating the fluid ports in the sides near the bottom as well as the bottom surface of the flexible bag. The fluid ports may be located at the same or different elevations within the flexible bag. Some of these fluid ports may be located below the fluid surface contained in the flexible bag or above the fluid surface contained in the flexible bag.

The fluid ports that are integrated into the flexible bag may include a connector allows conduit or tubing to be easily connected. For example, the fluid ports may include a barbed end that interfaces with an end of the conduit or tubing. In some embodiments, the fluid ports may be configured to direct the returning flow into the interior of the flexible bag in a directional manner. For example, the orientation of the fluid ports may be oriented to create a mixing or turbulent fluid environment within the interior of the flexible bag. In some embodiments, valves are used to prevent fluid contained in the flexible bag from exiting the flexible bag. For example, after fluid has been recirculated to the flexible bag via the ports, the valves of the fluid ports may be closed (either by manual operation or through the valve design (e.g., check-valve). The conduit or tubing can then be removed from the flexible bag.

In one embodiment, a single-use flexible fluid container includes a flexible bag having a bottom surface and one or more side surfaces, the bottom surface comprising a sump region, the sump region located in a central region of the bottom surface of the flexible bag, the sump region defined by a bottom sump surface and one or more side sump surfaces that extend from the bottom surface of the flexible bag. A plurality of fluid ports disposed in the one or more side surfaces or the bottom surface of the flexible bag. A first connecting port is disposed in the bottom sump surface and.

In another embodiment, a system for mixing fluids includes a flexible bag having a bottom surface and one or more side surfaces, the bottom surface comprising a sump region, the sump region located in a central region of the bottom surface of the flexible bag, the sump region defined by a bottom sump surface and one or more side sump surfaces that extend from the bottom surface of the flexible bag. A plurality of fluid ports are disposed in the one or more side surfaces or the bottom surface of the flexible bag. A first connecting port is disposed in the bottom sump surface and a second connecting port is disposed in the one or more side sump surfaces. A first pump is secured to the first connecting port, the first pump having an inlet and one or more outlets. A second pump is secured to the second connecting port, the second pump having an inlet and one or more outlets, wherein one or more outlets from the first pump and/or the second pump are connected via conduit or tubing to one of more of the plurality of fluid ports.

### BRIEF DESCRIPTION OF THE DRAWINGS

FIG. 1A is a front view of a single-use flexible bag according to one embodiment.

FIG. 1B is a side view of the single-use flexible bag of FIG. 1A.

FIG. 1C is a rear view of the single-use flexible bag of FIG. 1A.

FIG. 1D is a front view of a single-use flexible bag with cross-section line E-E′.

FIG. 1E is a cross-sectional view of the single-use flexible bag taken along the line E-E′ of FIG. 1D.

FIG. 1F is a front view of a single-use flexible bag according to another embodiment.

FIG. 1G is a side view of the single-use flexible bag of FIG. 1F.

FIG. 2A is a front view of the single-use flexible bag of FIG. 1A secured to two pumps via ports located in the sump region.

FIG. 2B is a side view of the single-use flexible bag of FIG. 1A secured to two pumps via respective ports.

FIG. 2C is a rear view of the single-use flexible bag of FIG. 1A secured to two pumps via respective ports.

FIG. 2D is a front view of a single-use flexible bag with cross-section line E-E′

FIG. 2E is a cross-sectional view of the single-use flexible bag taken along the line E-E′ of FIG. 2D.

FIG. 2F is a front view of the single-use flexible bag of FIG. 1A secured to two pumps via respective ports.

FIG. 2G is a perspective view of the single-use flexible bag of FIG. 1A secured to two pumps via respective ports.

FIG. 3A is a front view of a single-use flexible bag with cross-section line B-B′.

FIG. 3B is a cross-sectional view of the single-use flexible bag taken along the line B-B′ of FIG. 3A.

FIG. 3C is a front view of a single-use flexible bag with cross-section line D-D′.

FIG. 3D is a cross-sectional view of the single-use flexible bag taken along the line D-D′ of FIG. 3C.

FIG. 3E is a front view of a single-use flexible bag with detail F.

FIG. 3F is an enlarged view of detail F of FIG. 3E.

FIG. 4 is another embodiment of a flexible bag with attached pumps.

FIG. 5 illustrates a side view of a single-use flexible bag secured to first and second pumps. Conduit or tubing is connected to an outlet of one or both of the pumps and returns fluid to the interior of the flexible bag via one or more fluid port(s).

### DETAILED DESCRIPTION OF THE ILLUSTRATED EMBODIMENTS

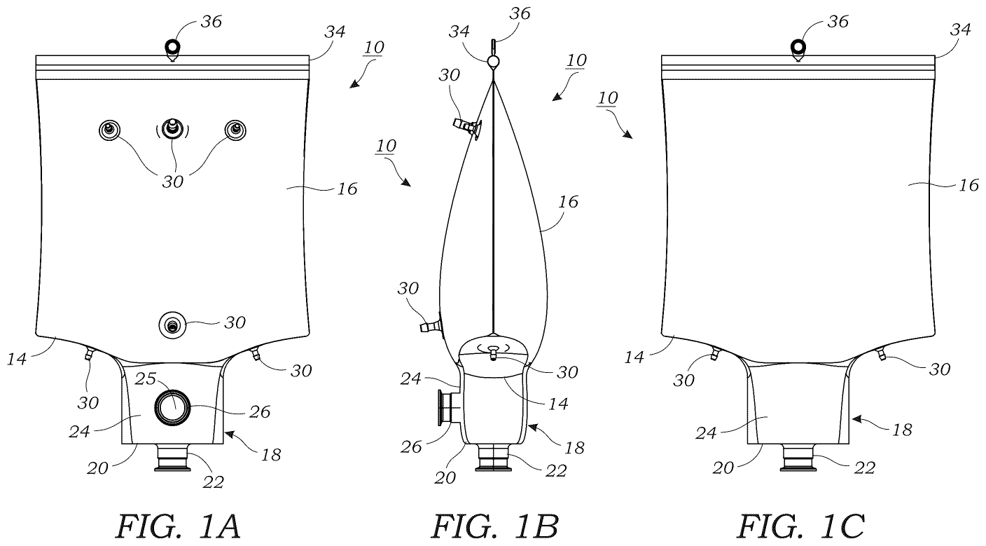

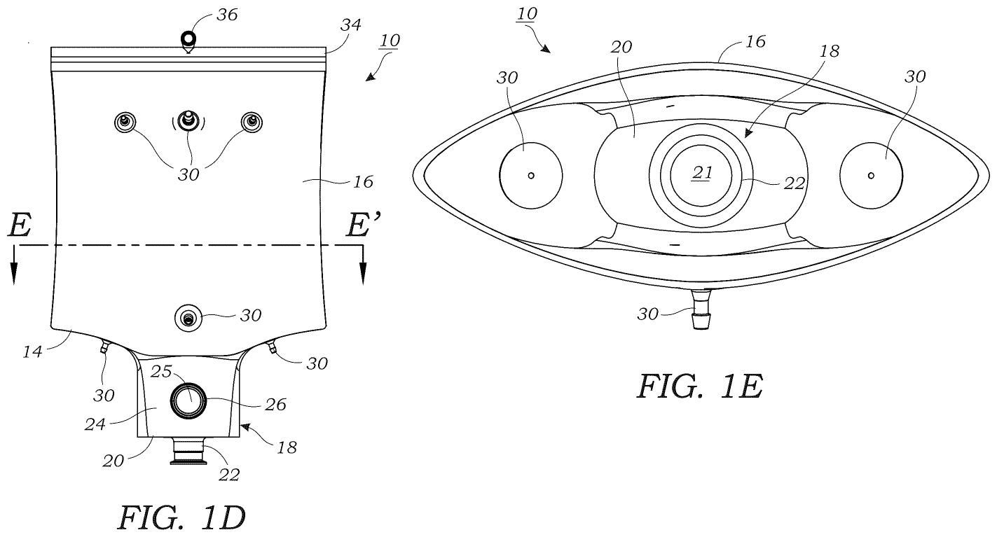

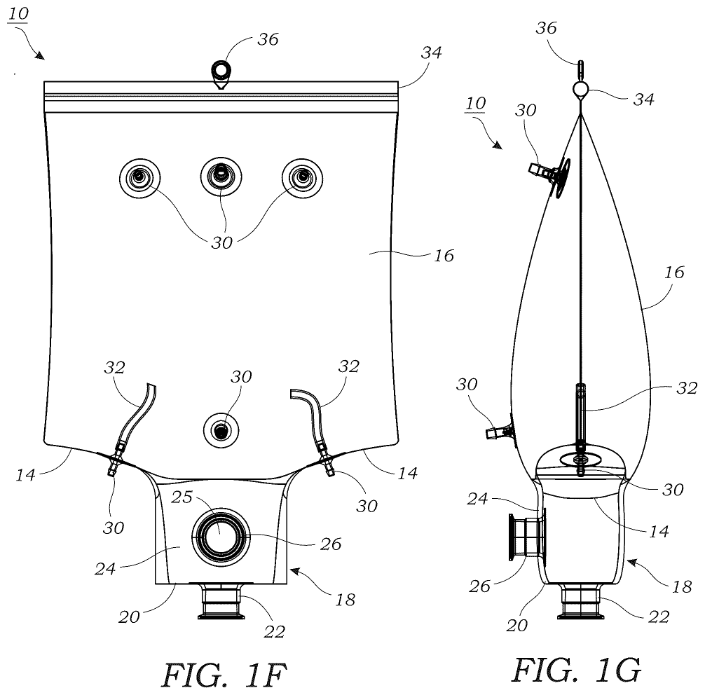

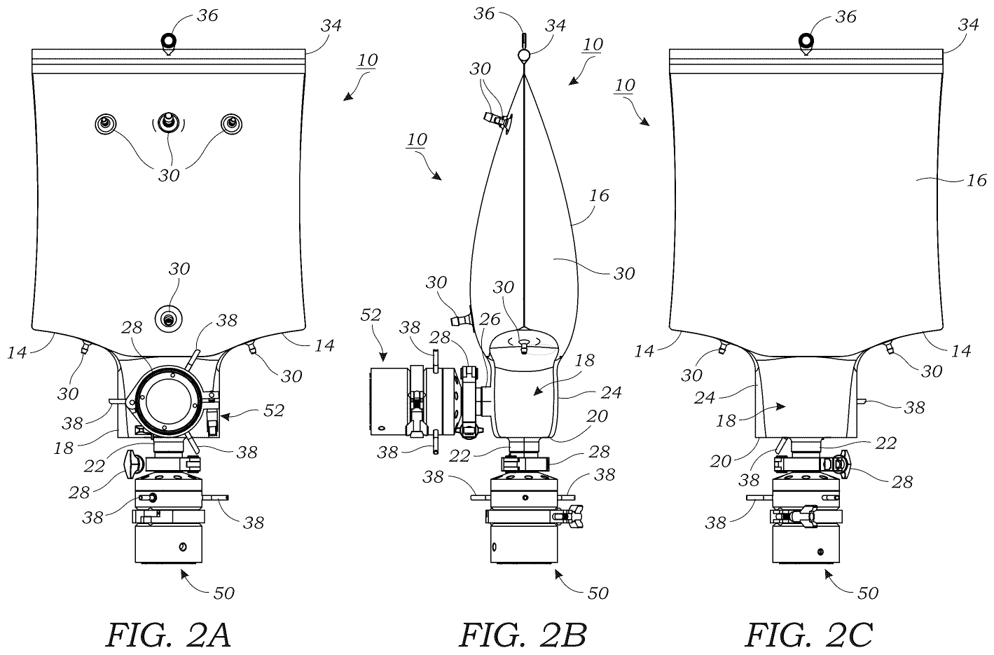

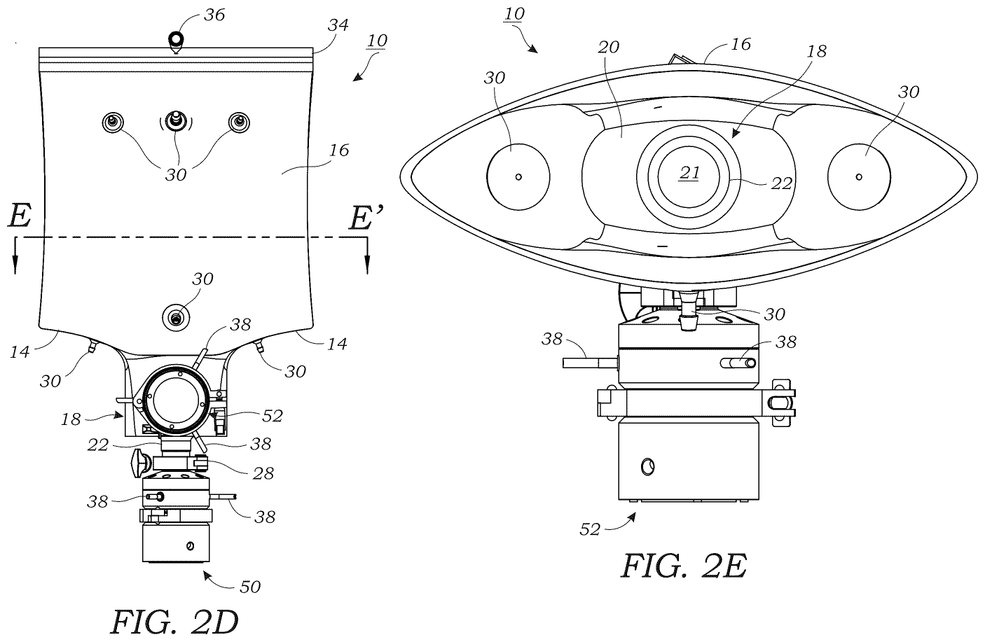

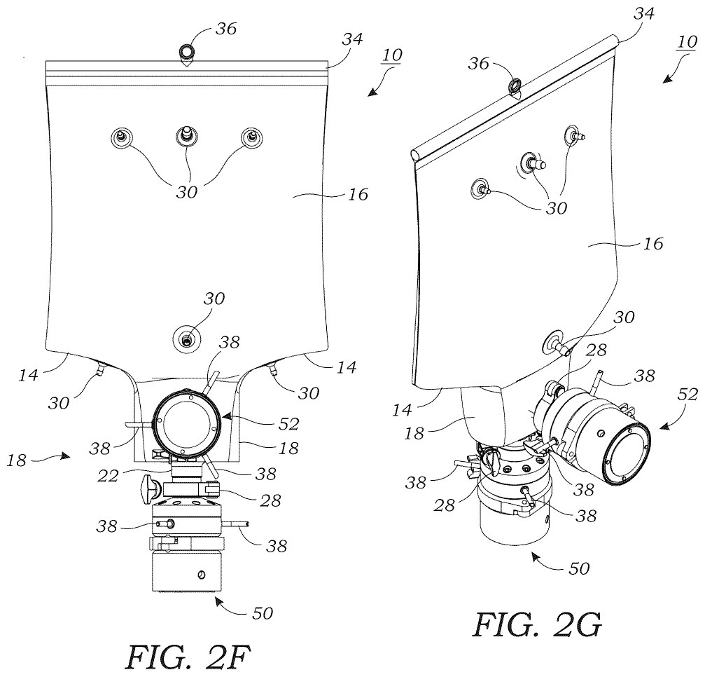

FIGS. 1A-1E illustrates one embodiment of the single-use flexible container 10 in the form a flexible bag (referred to as flexible bag 10). The flexible bag 10 has a bottom surface 14 and one or more side surfaces 16. In some embodiments, the flexible bag 10 may also have a top surface (not shown). The bottom surface 14 that includes a sump region 18. The sump region 18 of the flexible bag 10 is located generally at the central region of the bottom surface 14 and further extends in the downward direction (based on the orientation of the flexible bag 10 when being used). The bottom surface 14 adjacent to the periphery of the sump region 18 is tapered toward the sump region 18 so that when fluid is evacuated from the interior of the flexible bag 10 fluid will naturally flow, due to gravity, into the sump region 18. With reference to FIGS. 1A-1D, in one embodiment, the sump region 18 includes one or more side sump surfaces 24 that extend away from the bottom surface 14 of the flexible bag 10. The one or more side sump surfaces 24 terminate in a bottom sump surface 20 of the sump region 18. The bottom sump surface 20 includes an aperture 21 formed therein (FIGS. 1E, 2E, 3B, 3D). In one embodiment, a connecting port 22 is located on or in the aperture 21 and is used to connect with other components such as, for example, a pump 50 (as seen in, for example, FIGS. 2A-2D). In one embodiment, the connecting port 22 is a flanged port or a port used with a sanitary connector. The connecting port 22 is formed integral with the flexible bag 10 or alternatively bonded or welded to the flexible bag 10.

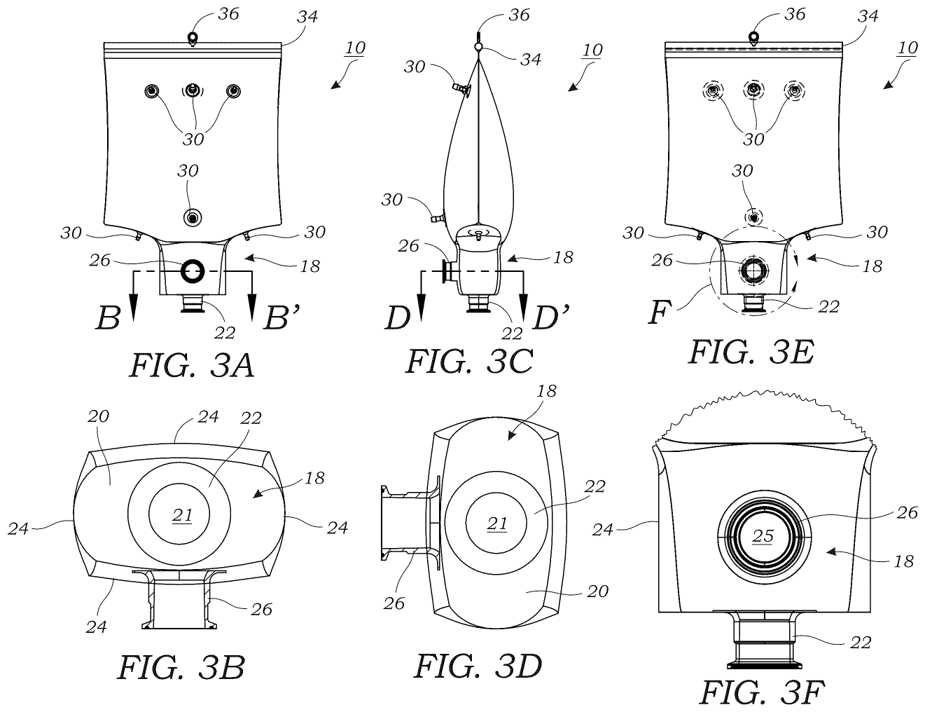

With reference to FIGS. 1A-1D, IF, 3F, in one embodiment, the sump region 18 includes an aperture 25 formed in the one or more side sump surfaces 24 of the sump region 18 (FIGS. 1A, 1D, IF, 3F) that that includes another optional connecting port 26. This connecting port 26, like port connecting 22, is used to connect with other components such as a pump 52 (as seen in FIGS. 2A, 2B, 2D, 2E, 2F, 2G, 4, and 5). The connecting port 26 may be formed integrally with the flexible bag 10 or alternatively bonded or welded to the flexible bag 10. The connecting ports 22, 26 may include a variety of sizes (e.g., 1.5 inches, 3 inches, 4 inches, and the like). The connecting ports 22, 26 may be the same size as each other or different. For example, connecting port 26 that exists on the one or more side sump surfaces 24 may include a smaller size which accommodates a smaller-sized pump 52. The connecting ports 22, 26 may include flanged ports or a port with a sanitary connector or end may be secured to using mating flanged surface on the pumps 50, 52 as illustrated in FIGS. 2A-2G. The two components (e.g., connecting ports 22, 26 and pumps 50, 52) may be secured to one another via a sanitary clamp 28 or the like as seen in FIGS. 2A-2D, 2F, 2G). In the flexible bag 10 embodiment illustrated herein, there are four (4) side sump surfaces 24 of the sump region 18 (best seen in FIGS. 3A and 3B) although it should be appreciated that more or less discrete surfaces may be used. For example, there may be a single cylindrical-shaped side sump surface 24; although this may make it more complicated to manufacture the included connecting port 26. The one or more side sump surfaces 24 and the bottom sump surface 20 of the sump region 18 define the volume of the sump region 18, which may vary depending on the particular flexible bag 10 construction or application. As stated herein, the bottom surface 14 of the flexible bag 10 tapers downward (c.g., FIGS. 1A, 1C) toward the sump region 18 so that any fluid in the flexible bag 10 will flow into the sump region 18. This taper of the bottom surface 14 may be a continuous taper such as that illustrated in FIG. IF or it may be a straight taper.

The flexible bag 10 includes a plurality of fluid ports 30 disposed in one or more side surfaces 16 of the flexible bag 10. In addition, the flexible bag 10 may include additional fluid ports 30 disposed in the bottom surface 14 such as illustrated in FIGS. 3A, 3C, and 3E. In some embodiments, the fluid ports 30 may have optional valves (e.g., check-valves or the like) incorporated therein. As seen in FIGS. IF, 1G, in one optional configuration, the fluid ports 30 located in the bottom surface 14 may include optional tubing segments 32 that are located inside the flexible bag 10. The tubing segments 32 may aid in fluid mixing within the flexible bag 10 when fluid is pumped into the flexible bag 10 via the fluid ports 30 as the tubing segments 32 undulate and thrash about inside the interior of the flexible bag 10. Of course, these tubing segments 32 are optional and may be omitted.

The fluid ports 30 may be located on the side surfaces 16 and/or the bottom surface 14. The fluid ports 30 may be used to deliver fluid into the interior of the flexible bag 10. The fluid ports 30 may be located at various levels or heights of the flexible bag 10 including both above and below the level of the fluid contained within the flexible bag 10. As explained herein, in some embodiments, fluid is pumped out of the flexible bag 10 using one or more of the pumps 50, 52 and returns fluid to the flexible bag 10 via a flexible conduit or tubing 70 (FIG. 5) that connects to an outlet of the pump(s) 50, 52 and also to the fluid port(s) 30 (e.g., the fluid ports 30 operate as return ports). In this way, fluid contained in the flexible bag 10 can then be recirculated using the pump(s) 50, 52 back into the flexible bag 10 for mixing and/or agitation. The flexible bag 10 may further include one or more fluid ports 30 that are inlet ports that are used to input separate fluid into the flexible bag 10. These fluid ports 30 may also function as vents. In this case, the fluid is a gas.

In the embodiments illustrated herein, the top of the flexible bag 10 terminates at a seam that includes a hanger sleeve 34 that can accommodate a hanger (e.g., hanging rod). Eyelet(s) 36 may also be provided at the top of the flexible bag 10 to allow for mounting the flexible bag 10 from above (c.g., hung from a support). The flexible bag 10 is typically formed from a polymer material. The polymer material may include a single layer film or multi-layer film. In one example, flexible bag 10 is formed from a multi-layer film layer that includes a durable, ethylene-vinyl alcohol copolymer (EVOH) that is oriented on the outside (non-product contact) of the flexible bag 10 while the inside or product contact side of the flexible bag 10 is made from polyethylene (PE). The fluid ports 30 may be made from PE as well. The fluid ports 30 may be configured with barbed ends so that the conduit or tubing 70 can be secured thereto. In other embodiments, the flexible bag 10 may be formed from single layer fluoropolymers or multi-layer fluoropolymers. The fluid ports 30 may be made from fluoropolymers. The fluids ports 30 may be laser welded or otherwise secured to the flexible bag 10 (e.g., using adhesive or the like).

FIGS. 2A-2G illustrate flexible bag 10 of FIGS. 1A-1E attached to respective pumps 50, 52. Each pump 50, 52 is connected at their inlets to the respective connecting ports 22, 26 and secured with respective sanitary clamps 28. In the illustrated embodiment, each pump 50, 52 has multiple outlets 38 (three such outlets illustrated). Some of these outlets 38 may be fluidically coupled via conduit or flexible tubing 70 back to the flexible bag 10 as explained above and illustrated in FIG. 5. Specifically, flexible tubing or other conduit 70 is connected to the outlet(s) 38 at one end and connected to the fluid port(s) 30 at the other end. In this manner, fluid is pumped one or more of the pumps 50, 52 back into the flexible bag 10. For example, pump 52 that is mounted to the side sump surface 24 of the sump region 18 may be used to recirculate fluid back to the flexible bag 10 while the other pump 50 is used to pump fluid from the flexible bag 10 to another location or process. While multi-outlet pumps 50, 52 are illustrated in FIGS. 2A-2G, in other embodiments, a pump 50, 52 with only a single outlet 38 may be used. In addition, the pump(s) 50, 52 may include inlets (not shown) therein that are used to input other fluids into the pump(s) 50, 52. These inlets may also be used to fill the flexible bag 10 via outlet(s) 38 from the pump(s) 50, 52.

The flexible bag 10 is preferably a single-use or disposable bag that can be discarded after use. The flexible bag 10 may be provided to the end user in an empty state and filled as desired. Alternatively, the flexible bag 10 may, in some embodiments, be prefilled with fluid. In other embodiments, the flexible bag 10 may be empty and then filled with the one or more pumps 50, 52 and then removed from the pump 50, 52 with the liquid inside.

To use the flexible bag 10, the flexible bag 10 is hung via the hanger sleeve 34 and eyelet 60 and the connecting ports 22, 26 are secured to the respective pumps 50, 52 using, for example, the sanitary clamp 28. Given the weight of the pumps 50, 52, the pumps 50, 52 and possibly the flexible bag 10 are supported in a rack, mount, fixture, or housing (not shown). Fluid may be loaded into the flexible bag 10 using one or more fluid ports 30. Fluid may, in some embodiments, be input into the flexible bag 10 via the pump(s) 50, 52. After filing the flexible bag 10 with fluid, one or more of the pumps 50, 52 is then used to pump the fluid from inside the flexible bag 10 through one or more of the outlets 38. The outlets 38 may be coupled to conduit or tubing 70 that are used to direct fluid flow as seen in FIG. 5. Some or all of these conduits or tubing 70 may return back to the flexible bag 10 where they interface with the fluid ports 30. For example, pump 52 may be used primarily or exclusively as a recirculating pump to recirculate fluid back into the flexible bag 10. The other bottom pump 50 may be used to pump fluid to other downstream operations. However, the bottom pump 50 may also be used for recirculation. For example, pump 50 may include multiple outlets 38 and some of these outlets 38 may return back to the flexible bag 10. The pumps 50, 52 may operate sequentially or simultaneously. In addition, valves may be optionally located in the different fluid paths or outlets 38 so that may be selectively opened or closed as necessary given the particular use. After use, the various segments of conduit or tubing 70 may be removed from the respective fluid ports 30 and the flexible bag 10 may be detached from the pumps 50, 52. The flexible bag 10 can then be discarded.

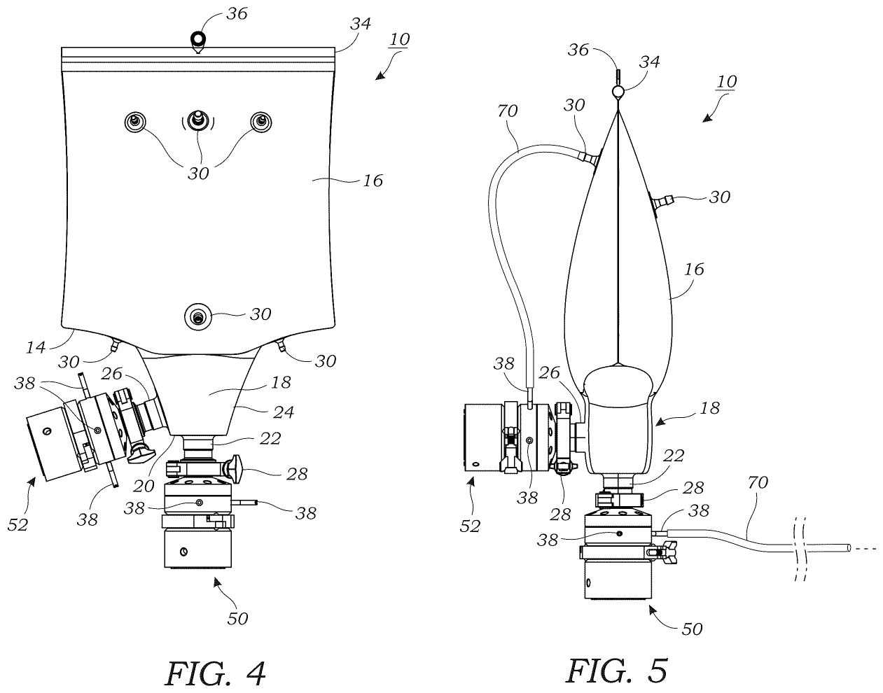

FIG. 4 illustrates another embodiment of a flexible bag 10. In this embodiment, the sump region 18 is less pronounced and the pump 52 that is attached to connecting port 26 is optimized in its location to place the same as low as possible on a side sump surface 24 within the sump region 18 but not on the bottom surface 20. Here, the connecting port 26 and the attached pump 52 are located close to or adjacent to the bottom sump surface 20 of the sump region 18. This allows agitation and mixing via the pump 52 in a very small volume. As seen in FIG. 4, the attached pump 52 may be oriented at a non-perpendicular angle (with respect to the vertical axis of the pump 50) depending on the configuration although other orientations of the pump 52 are contemplated. In particular the side sump surfaces 24 are angled and not perpendicular to the bottom sump surface 20 of the sump region 18.

While the flexible bag 10 disclosed herein is illustrated as containing two pumps 50, 52, it should be appreciated that in other embodiments, there may be a single pump 50 that is used in connection with the flexible bag 10. In this embodiment, the second connecting port 26 is omitted, capped, or otherwise closed. For example, a cap or lid may be secured to the connecting port 26 using a clamp 28 or the connecting port 26 maybe omitted entirely. Further, while embodiments of the present invention have been shown and described, various modifications may be made without departing from the scope of the present invention. For example, the types of flexible bags 10 that can be used may vary beyond those specifically recited herein. These include, flexible bags 10 that include a top surface. The invention, therefore, should not be limited, except to the following claims, and their equivalents.

What is claimed is:

1. A single-use flexible fluid container comprising:
a flexible bag having a bottom surface and one or more side surfaces, the bottom surface comprising a sump region, the sump region located in a central region of the bottom surface of the flexible bag, the sump region defined by a bottom sump surface and one or more side sump surfaces that extend from the bottom surface of the flexible bag;
a plurality of fluid ports disposed in the one or more side surfaces or the bottom surface of the flexible bag; and
a first connecting port disposed in the bottom sump surface.

2. The single-use flexible fluid container of claim 1, further comprising a first pump having an inlet and one or more outlets, wherein the first pump is secured to the first connecting port.

3. The single-use flexible fluid container of claim 2, further comprising a second connecting port disposed in the one or more side sump surfaces.

4. The single-use flexible fluid container of claim 3, further comprising a second pump having an inlet and one or more outlets, wherein the second pump is secured to the second connecting port.

5. The single-use flexible fluid container of claim 4, wherein the second pump comprises a plurality of outlets and wherein at least some of these outlets are connected via conduit or tubing to one of more of the plurality of fluid ports.

6. The single-use flexible fluid container of claim 3, wherein the second connecting port is located in the one or more side sump surfaces at a location that is adjacent to the bottom sump surface.

7. The single-use flexible fluid container of claim 6, wherein the one or more side sump surfaces containing the second connecting port is angled in a non-perpendicular arrangement relative to the bottom sump surface.

8. The single-use flexible fluid container of claim 1, wherein the flexible bag further comprises a top surface.

9. The single-use flexible fluid container of claim 1, wherein the bottom surface comprises a tapered surface that surrounds a periphery of the sump region.

10. The single-use flexible fluid container of claim 9, wherein the tapered surface is a straight tapered surface.

11. The single-use flexible fluid container of claim 9, wherein the tapered surface is a continuous tapered surface.

12. A system for mixing fluids comprising:
a flexible bag having a bottom surface and one or more side surfaces, the bottom surface comprising a sump region, the sump region located in a central region of the bottom surface of the flexible bag, the sump region defined by a bottom sump surface and one or more side sump surfaces that extend from the bottom surface of the flexible bag;
a plurality of fluid ports disposed in the one or more side surfaces or the bottom surface of the flexible bag;
a first connecting port disposed in the bottom sump surface;
a second connecting port disposed in the one or more side sump surfaces;
a first pump secured to the first connecting port, the first pump having an inlet and one or more outlets, and
a second pump secured to the second connecting port, the second pump having an inlet and one or more outlets, wherein one or more outlets from the first pump and/or the second pump are connected via conduit or tubing to one of more of the plurality of fluid ports and wherein the first pump and second pump simultaneously pump fluid from the flexible bag.

13. The system of claim 12, wherein at least one of the first pump or the second pump has one or more additional inlets.

14. The system of claim 12, wherein the flexible bag further comprises a top surface.

15. The system of claim 12, wherein the bottom surface comprises a tapered surface that surrounds a periphery of the sump region.

16. A method of using the single-use flexible fluid container of claim 1, further comprising securing a first pump to the first connecting port.

17. The method of claim 16, further comprising actuating the first pump to recirculate at least a portion of a fluid contained in the flexible bag back to the flexible bag via one of the plurality of fluid ports.

18. A method of using the single-use flexible fluid container of claim 3, further comprising securing a second pump to the second connecting port.

19. The method of claim 18, further comprising actuating the second pump to recirculate at least a portion of a fluid contained in the flexible bag back to the flexible bag via one of the plurality of fluid ports.
